(19) United States
(12) Patent Application Publication
(10) Pub. No.: US 2023/0273157 A1
(43) Pub. Date: Aug. 31, 2023

(54) METHODS AND SYSTEMS FOR DETECTING PARTICLE OCCUPANCY

(71) Applicant: LUMICKS CA HOLDING B.V., Amsterdam (NL)

(72) Inventor: Felix Oswald, Amsterdam (NL)

(21) Appl. No.: 18/028,155

(22) PCT Filed: Sep. 23, 2021
(86) PCT No.: PCT/NL2021/050575

§ 371 Date: Mar. 23, 2023

(30) Foreign Application Priority Data
Sep. 24, 2020 (NL) 2026531

(51) Int. Cl. G01N 29/036; G01N 29/22; G01N 33/50

(52) U.S. Cl. CPC G01N 29/036; G01N 29/222; G01N 33/5005; G01N 2291/014; G01N 2291/0257

(57) ABSTRACT

A method comprises providing a sample holder having a holding space for a sample comprising particle(s) in a fluid medium, the holder comprising a wall providing a wall surface portion, and providing a signal generator for generating an acoustic wave in the holder; providing using the signal generator a driving signal to the holder generating a standing longitudinal acoustic wave in the holder comprising at least one of a node and an antinode; and determining an acoustic resonance frequency characteristic. The method further comprises: providing a sample comprising particle(s) in a fluid medium in contact with the wall surface portion; determining a variation in the resonance frequency characteristic of the holder; determining a difference in position of one or more of the particles with respect to a node and/or an antinode of the acoustic wave and/or the wall surface portion, in particular contact, attachment and/or adhesion.

### CROSS-REFERENCE TO RELATED APPLICATION(S)

This application is a Section 371 National Stage Application of International Application No. PCT/NL2021/050575, filed Sep. 23, 2021, and published as WO 2022/066011 A1 on Mar. 31, 2022, and further claims priority to Netherlands Patent Application No. 2026531, filed Sep. 24, 2020.

### TECHNICAL HELD

The present disclosure relates to methods and systems for studying particles

### BACKGROUND

The study of cell interactions, e.g. the binding strength of cells on cells is a highly relevant and active research area in biosciences. For example, the avidity characterizes the cumulative effect of multiple individual binding interactions between cells. Similarly, the affinity characterizes the strength with which one molecule binds to another molecule, e.g. the strength with which a receptor on the cell membrane of an immune cell binds to an antigen on the target cell. The avidity and affinity are examples of parameters that play an essential role in the study and development of therapies in medicine, e.g. immune oncology.

A technique for studying interactions between cells is referred to as force spectroscopy. For example, WO 2018/083193 describes a so-called acoustic force spectroscopy AFS system that is configured to examine interactions between cells by applying a force to the cells. The system includes a microfluidic cell comprising a functionalized wall surface which may include target cells. A plurality of unlabelled effector cells, e.g. T-cells, can be flushed into the microfluidic cell, so that they can settle and bind to target cells. Thereafter, an acoustic source is used to exert a ramping force on the bound effector cells so that effector cells will detach from the target cells at a certain force. During this process, the spatiotemporal behavior of the effector cells in the microfluidic cells is imaged using an imaging microscope. The interaction between cells, e.g. the force at which the effector cells detach, may be determined by analysing the captured (video) images. For example, the cell avidity of the effector cells can be determined this way.

Desires remain for such techniques e.g. for another detection method and/or detection principle which preferably is combinable with the optical methods described above; for increases in one or more of efficiency, speed, versatility, applicability, precision; and for reductions in complexity, in required computing power and/or in operating costs.

Reference is made to US 2013/218048 A1 disclosing an apparatus and method for determining a biochemical function of a fluid; WO 2005/001440 A1 disclosing a crystal sensor; US 2004/150296 A1 disclosing a material sensing sensor and module using thin film bulk acoustic resonator; US 2017/122912 A1 disclosing a fluidic device with fluid port orthogonal to functionalized active region; and US 2007/220970 A1, disclosing a measuring cell as well as constructing methods for a measuring cell and measuring apparatus with a mount for such a measuring cell.

### SUMMARY

In view of the above, a method is provided comprising the steps of:
providing a sample holder comprising a holding space for holding a sample comprising one or more particles in a fluid medium, wherein the sample holder comprises a wall providing a wall surface portion in the holding space, and
providing a signal generator for generating an acoustic wave in the sample holder;
providing using the signal generator a driving signal to the sample holder generating a standing longitudinal acoustic wave in the sample holder comprising at least one of a node (N) and an antinode in the holding space, the signal having a signal frequency, a signal amplitude and a signal power; and
determining an acoustic resonance frequency characteristic of the sample holder for the acoustic wave.

The method further comprises the steps of:
providing a sample comprising one or more particles in a fluid medium in the holding space, in particular being in contact with the wall surface portion;
determining a variation in the resonance frequency characteristic of the sample holder;
determining on the basis of the variation a difference in position of one or more of the particles with respect to at least one of a node of the acoustic wave, an antinode of the acoustic wave and the wall surface portion, in particular at least one of contact, attachment and adhesion of one or more of the particles to the wall surface portion.

The resonance frequency characteristic of the sample holder may comprise one or more of the resonance frequency, a frequency width of a resonance peak about the resonance frequency and a resonance quality factor.

A resonance frequency is in principle a system parameter, in particular a characteristic of the oscillator, the sample holder and the sample (sample medium and any particles therein) and (mechanical) properties of these components.

It has been found that determination of a resonance frequency characteristic may be very accurate and that small deviations may be determined and that such deviations may be attributed to small changes in the system. In particular, it has been found that particles having an acoustic index differing from the sample fluid and/or from the wall tend to affect the acoustic properties of the sample holder comprising the sample such that a position shift of such particles with respect to the acoustic wave and/or the wall portion may detectably affect the acoustic resonance properties of a sample holder. In particular, contact of particles with the wall surface portion may be determined. Thus, resonance frequency measurements allow studying behavior of the particles in the sample holder.

The variation may depend on an acoustic index difference from one or more material properties of the sample fluid, the wall and one or more surface layers on the wall surface portion.

The determination of a difference in position may be time resolved, which may provide at least an indication of a movement and/or a velocity of the one or more particles.

Contact with the wall surface portion may e.g. be due to one or more of gravity, adhesion, biological (re-) action, (bio-) chemical reaction, (bio-) physical action and any force applied to the particles and/or to the sample holder.

The acoustic wave may be an ultrasound wave, e.g. having a frequency in a range of 1-30 MHz, preferably in a range 5.20 MHz such as 7-15 MHz. Preferably, the wavelength of the acoustic wave in the sample holder is on the order of a size of the particles such as a diameter, e.g. 240 times the particle size, preferably 8-20 times the particle diameter. The acoustic wave may be a standing wave, at least during part of the method. The acoustic wave may be a longitudinal bulk wave and may be arranged for applying an acoustic force to the one or more particles in the sample fluid. Applying an acoustic force to the one or more particles may form (part of) a step of the method, wherein the acoustic force may be provided at a strength sufficient to move one or more particles and/or to counteract another force on the one or more particles. Typical wavelengths for use with biological cells may be about 100-200 micrometer. E.g. in an embodiment exploiting acoustic forces (see below) an acoustic wavelength of ca. 160 micrometer and having an antinode of a resonant wave at or near the wall surface portion a cell of about 8 micrometer diameter may be lifted from the wall surface to a nodal plane at about 40 micrometer from the wall surface.

The sample holder may comprise an oscillator, which may comprise a transducer for converting an electrical driving signal, such as an oscillating voltage, into an acoustic signal in the sample holder providing the acoustic wave.

Determination of a resonance frequency may comprise performing a frequency sweep over a range of frequencies comprising at least one resonance frequency and detecting the at least one resonance frequency. System behavior may then be further studied employing frequencies in a comparably narrow frequency range about the resonance frequency. An expected resonance frequency may be calculated, facilitating the determination.

The resonance quality factor, also referred to as quality number or Q-factor, provides an indication of frequency characteristics and an indication of efficiency of exciting the acoustic wave at and/or near the resonance frequency. Therewith, the Q-factor provides (additional) information about behavior of at least part of (a sample in) the sample holder.

The acoustic resonance frequency of the sample holder may be determined with or without at least a portion of a sample in (the holding space of) the sample holder. Determination without a sample may provide information regarding the sample holder per se and allow determination of one or more aspects of the sample itself, e.g. the determination without a sample providing a background/baseline reference. The portion of the sample may comprise a sample fluid without particles, e.g. for provision of a reference, e.g. a further reference. Any such determination may be in dependence of at least one of signal power, temperature of at least part of the sample holder and/or the sample, time and sample composition.

Thus, the sample holder and/or resonance frequency may be characterized in view of experimental conditions, for instance.

Determining a variation in the resonance frequency characteristic of the sample holder may comprise providing the driving signal a number of times, determining a variation relation between the resonance frequency of the sample holder and at least one of time, the signal power, and, if applicable, the number of times of providing a driving signal.

The method may then comprise determining on the basis of the variation relation the difference in position of one or more of the particles. Thus, characteristics of at least part of the sample may be determined. Time-dependent measurements may, e.g. allow determination of settlement and/or adhesion reactions to the wall surface portion and/or evolutions within at least part of the sample. Determination as a function of signal power, related to acoustic power, enables e.g. varying signal to noise levels and/or probing characteristics that may be dependent on one or more of force, size and mass. Signal power may be varied as a function of time within one instance of providing a driving signal, i.e. provision of an acoustic wave, and/or between different instances of providing a driving signal.

The driving signal may be provided one time, e.g. in case of a continuous measurement, or several times, e.g. for repeated and/or interrupted measurements. Determination as a function of the number of times of providing the driving signal facilitates determination of evolutions due to and/or between different instances of providing a driving signal.

The method may comprise changing at least part of the sample and/or allowing at least part of the sample to change as a function of at least one of time, temperature, illumination, sample composition, and flow of at least part of the sample fluid, thus providing a sample change.

Determining a variation in the resonance frequency of the sample holder may comprise determining a change relation between the resonance frequency of the sample holder and the sample change.

The method may then comprise determining on the basis of the change relation the difference in position of one or more of the particles.

This allows (determination and/or study of) one or more of settling of the particles on the wall surface portion, temperature dependent properties which may be reversible or not, biological (re-) action, (bio-) chemical (re-) action, (bio-) physical (re-) action, etc. Biological particles may comprise cellular bodies and/or (parts of) organisms responding to light such as illumination intensity and/or illumination of color, e.g. by performing or halting biological processes and/or moving away from or rather towards the light.

Changing a sample composition may comprise introducing and/or removing at least some of the particles. Also or alternatively it may comprise introducing or removing one or more of a reactant, a feedstuff, and a biological interaction moiety for a biological sample component contained in at least part of the sample.

Providing a driving signal may comprise including the sample holder in an electric circuit and providing at least part of the signal as an electrical signal, preferably an oscillating signal such as an AC-signal generating the acoustic wave.

The method may then further comprise selecting at least one property of the driving signal and/or the sample holder selected from a group consisting of a voltage drop, an impedance, an admittance, a susceptance, a conductance and a signal phase shift, wherein the impedance may comprise one or more of a resistance, a capacitance and an inductance and may be a complex impedance;

determining a value of the selected property at a plurality of signal frequencies in a signal frequency range; and

determining on the basis of at least the selected property the resonance frequency and/or a quality factor for the sample holder with respect to the resonance frequency.

Such method facilitates detection of the resonance frequency and/or the resonance quality factor.

The impedance, admittance, susceptance, conductance and signal phase shift may be determined as one or more electrical properties of the sample holder and/or may include in the determination at least part of an oscillator, e.g. a transducer, for providing the periodic driving signal, which oscillator may be comprised in the sample holder. This facilitates detection and/or processing the respective selected property.

Determining a value of the selected property at a plurality of signal frequencies in a signal frequency range can be done by sweeping the frequency applied to the transducer in time while measuring the selected property and/or, for example, by applying a short burst containing a plurality of desired spectral frequencies simultaneously and measuring a response function of the sample holder. E.g. one may determine the impulse response of the sample holder after excitation with a suitably selected driving pulse.

Any method embodiment herein may comprise providing a driving signal with a single frequency or with a (periodic or aperiodic) modulated frequency such as one or more of a sum of plural frequencies, a frequency sweep and a chirp. Also, in some cases driving signals of different waveforms may be applied and/or combined with other waveforms (sinusoidal, triangular, saw tooth, etc.) which may allow studying different resonance behaviors.

At least one embodiment may comprise superposing a modulation frequency on the signal frequency, thus providing a modulated signal frequency; determining a frequency difference between the resonance frequency and at least one of the signal frequency and the modulated signal frequency. Such method may then further comprise adjusting the modulation frequency and/or the signal frequency in dependence of the frequency difference.

Such method steps may be repeated one or more times wherein the resonance frequency may be determined anew one or more times. Thus, any changes may be detected.

Such method may suitably comprise the further step of adjusting the signal frequency on the basis of the feedback signal. Thus, the signal frequency may be maintained at or near the resonance frequency, and in case of repetition also when the resonance frequency varies.

The determination of the frequency difference and the determination of the resonance frequency may be based on the selected property as indicated above; preferably then both are determined on the same selected property.

In particular, such method may comprise superposing a modulation frequency on the signal frequency thus providing a modulated signal frequency, wherein the step of determining a frequency difference between the signal frequency and the resonance frequency comprises determining the frequency difference with respect to the modulated signal frequency and the resonance frequency, and wherein the method further comprises adjusting the signal frequency and/or the modulation frequency in dependence of the frequency difference and/or adjusting the signal frequency and/or the modulation frequency on the basis of the feedback signal, where applicable.

Thus, active locking of the signal frequency may be provided. Such locking facilitates a periodic redetermination of the signal frequency with respect to the resonance frequency and/or the other way around thus facilitating reestablishment of the resonance frequency and/or the Q-factor and according adaptation of the signal frequency. Hence, reliability of the method and any measurement results obtained with it may be increased.

The modulation frequency may be any periodic modulation, in particular a sinusoidal, triangular or a saw-tooth modulation pattern.

Adjusting the modulation frequency in dependence of the frequency difference and/or the feedback signal may allow adaptation of the modulated signal frequency to a change in position and/or other characteristics of the resonance peak, such as frequency width and/or asymmetry.

The acoustic wave may be a standing wave, and/or the acoustic wave may be oriented perpendicular to the wall surface portion. Also or additionally, the acoustic wave may provide a force gradient in a direction away from the wall surface portion into the holding space for urging at least some of the particles towards the wall surface portion and/or for urging at least some of the particles away from the wall surface portion and into the holding space the wall surface portion.

A standing wave provides a reliable determination of a resonance frequency. A longitudinal standing wave may provide an acoustic force which may allow manipulation and/or study of one or more portions and/or particles of the sample. A force gradient urging particles away from or towards the wall surface portion facilitates studying properties of the respective particles. Urging particles from the sample fluid towards the wall surface portion may, for example, enable measurement of an amount or number of particles in the sample.

An acoustic wave oriented perpendicular to the wall surface portion may in particular increase the desired detection of a difference in position of one or more of the particles.

Any method herein may comprise determining an acoustic force on at least some of the one or more particles at or near the wall surface portion. Any method herein may also or alternatively comprise determining an adhesion of one or more of the particles to the wall surface portion and/or may comprise determining an adhesion strength and/or a detachment force of the particle to the wall surface portion.

Determination of the acoustic force allows in particular better understanding of any dynamics (or not) of the particle. Determining adhesion may provide information indicative of processes affecting the adhesion. In particular in case of biological samples, many processes are associated with changes in shape and/or surface properties of such particles, so that knowledge of adhesion properties may provide information about such processes.

The wall surface portion may define at least part of the holding space, e.g. defining a cavity, or be part of a structure protruding into the holding space such as a divider. In particular, the sample holder may comprise an acoustic wave generator such as a transducer and/or a piezo crystal, etc. connected with the wall providing the wall surface portion. In some embodiments, an acoustic wave generator may be connected with a wall opposite the wall surface portion.

The wall surface portion may be provided with a functionalized wall portion. Also or alternatively at least some of the particles may be cellular bodies. Any of these allow studying particular interactions, as defined by the functionalization and/or the nature of the cellular bodies.

Interaction of one or more cellular bodies in the sample with the functionalized wall surface portion may be studied, e.g. a relation of adhesion of the cellular body to the functionalized wall surface portion and (the force of) the acoustic wave. Also or alternatively, such relation could be determined using to another force, as a suitable force could be applied using any other method, see below. The presently provided method allows studying bonding forces of cellular bodies to the functionalized wall portion; the entire contact surface of a cellular body with the wall portion may be probed at once. This relates to the amount of bonds and the bonding force per bond. Moreover, plural cellular bodies may be studied simultaneously which may provide statistic distribution information within one or a few measurements.

The cellular bodies may be cell portions like subcellular organelles, cell nuclei, and/or mitochondria. However, the cellular bodies may also be unicellular or pluricellular, such as small clumped cell groups, plant or animal biopts, dividing cells, budding yeast cells, colonial protists, etc. The cellular bodies may also be animal embryos in an early stage of development (e.g. the morula-stadium of a mammal, possibly a human embryo). In particular cases different types of cellular bodies may be studied together. E.g., cellular bodies from a mucosal swab, blood sample, or other probing techniques could be used.

One or more method embodiments herein enable studies on various properties of the one or more cellular bodies. Particular examples are presence, absence and/or quantification of abundance of biomolecules on the cellular bodies, surface adhesion forces and/or adhesion kinetics of the cellular bodies, to the functionalized surface portion, differences in any of the above under influence of biological processes active in the cellular bodies or induced by various chemical components that may be introduced into the sample.

Current cell-adhesion assays and methods that aim for analysing cell-surface biomolecule composition and abundance, require a large quantity of cells, are very laborious and depend on expensive instrumentation, e.g. requiring labelling and risking damage to the cell(s) to be studied (e.g. cell-lysis, antibody labelling). Furthermore, these techniques typically lack the ability to assess cell-adhesion forces and cell-adhesion kinetics, in particular at the single cell level. The presently provided method enables studies on the various properties of multiple individual cellular bodies in parallel, since multiple cellular bodies in the sample may contact and interact with the functionalized wall surface portion. This may increase accuracy of the study results and false positives or false negatives may be avoided.

The presently provided method enables studies on cellular bodies per se, without adhesion of foreign objects to the cellular bodies such as microbeads, magnets, chromophores, antibodies, various other labels etc. Thus, the cellular bodies may remain essentially unharmed by the present method and it is envisioned that after performing the method, the cellular bodies could be administered to a test subject and/or returned to a subject having donated the cellular bodies for studying; e.g. one or more of T-cells, leukocytes, erythrocytes and similar cellular bodies may be withdrawn from a subject, be studied in accordance with the method, and could thereafter be further analysed with various other methods (single-cell sequencing, fluorescence microscopy, cryo-electron microscopy, etc.) and/or administered to another subject (e.g. blood donation) or returned to the original subject itself. Smaller cellular bodies, e.g. taken from blood plasma, may also be studied prior to donation or return. Similarly, spermatozoa and/or eggs could be studied before artificial in-utero or in-vitro fertilisation, and fertilized eggs and/or first-stage embryos (e.g. morula or blastula stage) may be screened before implantation in a female subject for gestation.

Cellular adhesion and/or binding strength, which may involve specific antigen-receptor pairs, may be characterized. The functionalized wall surface may comprise specific antigens and/or interaction moieties. Suitable interaction moieties may comprise an antibody for selective binding to a particular target, e.g. a microbial cell or a cancer cell. E.g., specific antibodies exist for several major hospital infections. Such interaction moiety may normally be effective to deliver a conjugate of the interaction moiety to a predetermined pathological site in a mammal. A pathological site may comprise a target moiety which, together with the interaction moiety, constitutes a specific binding pair. In the present case the interaction moiety may be attached to the wall in the functionalized wall portion, e.g. by direct attachment or by forming the interaction moiety from or with a primer, and a binding pair may have sufficient binding force to adhere the cellular body to the functionalized wall portion. The interaction moiety may also be presented by an antigen presenting cell (APC) which may be a professional APC or any other cell presenting specific targets on its surface. The functionalized wall surface layer may comprise such cells; possibly as a cell monolayer. Cellular bodies in the sample may bind to the cells in the functionalized wall surface layer. Multiple different interaction moieties and multiple different receptors may be present on either the cells the functionalized wall surface layer or the cellular bodies in the sample and these may interact with each other sequentially or simultaneously and they may influence each other and show non-linear binding effects. The combined binding strength of such multiple interactions is commonly known as “avidity”. Dynamic aspects may also play a role as the binding of an antigen by one receptor may for example trigger a signalling pathway influencing other interaction moiety—receptor pairs and/or influencing cellular behavior in general. Thus, binding strengths may be influenced by e.g. incubation times and/or force applied during incubation, which influence may be detectable.

The interaction may also be based on bispecific antibodies (BsAbs)/bispecific monoclonal antibodies (BsMAbs), e.g. antibodies with two binding domains where one domain binds to specific moieties on cellular bodies in the sample and the other domain binds to specific moieties on the cellular bodies in the functionalized wall surface layer.

The interaction moiety may comprise an antibody, or an antibody fragment that binds to a cell-surface antigen, or a ligand or ligand fragment that binds specifically to a cell surface receptor. For instance, cancer cells usually have tumor associated antigens on their surface. Their complementary antibodies will bind very selectively to these tumor associated antigens. Ligand or ligand fragments however are also suitable. Various peptides are known to bind their cognate receptors with high affinity and thus would be suitable ligands for incorporation into the functionalized wall surface. Receptors are plasma membrane proteins which bind molecules, such as growth factors, hormones and neurotransmitters. Tumors develop from particular cell types which express certain subsets of these receptors. Taking advantage of this binding affinity between receptor and ligand enables target-specific studies and/or identification of cellular bodies.

Similarly, immune response relies on a complex interaction cascade between immune cells and their cell surfaces. For instance, B-cell activation depends on the binding of the B-cell receptor expressed on the B-cells surface to an antigen exposed on the surface of an antigen-presenting cell (APC). This in turn triggers a cascade of intracellular and intercellular events that leads to antibody secretion and pathogen attack by the complement system. Likewise, T-cell activation occurs via the interaction of an antigen on the surface of an APO with the T-cell receptor on the T-cell surface. Furthermore, T-cells recruitment to inflammatory/infected sites relies on the extravasation of T-cells from the bloodstream into tissue. Extravasation is initiated by a cytokine-regulated multistep adhesion process to the vascular epithelium followed by transmigration through the cell wall of blood vessels. Immunodeficiency and autoimmune diseases represent a misbalance in immune response. In all processes that can lead to abnormal immune response, e.g. altered lymphocyte activation, cell-adhesion, cell-migration and pathogen attack, the interaction of bio-molecules on the cell surface with binding partners in the extracellular environment is essential.

Representative examples of receptor-ligand pairs are set forth below:

| RECEPTOR | LIGAND |
|---|---|
| Epidermal growth factor receptor | epidermal growth factor (53 |
| | amino acids) |
| Platelet derived Growth factor receptor | platelet derived growth factor |
| Insulin like growth factor receptor | insulin-like growth factor |
| Glucagon growth factor receptor | glucagon (23 amino acids) |
| Vasopressin receptor | vasopressin (9 amino acids) |
| A thyroid stimulating Hormone receptor | thyroid stimulating hormone |
| Insulin receptor | insulin |
| T-cell receptor (TCR) | Peptide-MHC complex |
| Chimeric antigen receptor (CAR) | CD19 |

In an embodiment comprising a functionalized wall surface layer and cellular bodies as particles, one of the cellular bodies and functionalized wall surface layer may comprise effector cells and the other one of the cellular bodies and functionalized wall surface layer comprises target cells. Such embodiment may further comprise determining a binding characteristic of the effector cells to the target cells.

In a particular embodiment, one of the cellular bodies and functionalized wall surface layer comprises immune cells and the other one of the cellular bodies and functionalized wall surface layer comprises tumor cells. One particular example of the method is the accurate measurement of the intercellular binding strength of immune cells (e.g. T-Cells, CAR-T cells, NK cells, CAR-NK cells, etc.) to their target cells (e.g. tumor cells, virus infected cells, etc.).

Any embodiment of a method herein may comprise applying a non-acoustic force to the one or more particles, in particular in a direction away from or towards the wall surface portion.

A particular example comprises rotating at least part of the sample holder and applying a centrifugal force to the one or more particles.

The centrifugal force may in particular be used as a force towards or away from the wall surface portion, wherein the wall surface portion may be arranged in tangential and/or circumferential orientation relative to the axis of rotation about which the at least part of the sample holder is rotated.

Other examples comprise (use of) other types of force such as one or more of magnetic forces, electric forces (e.g. electrophoresis, dielectrophoresis) and optical forces (e.g. optical tweezers).

Such forces may allow manipulation of at least part of the particles while the acoustic resonance frequency characteristic is used to determine a difference in position of one or more of the particles with respect to at least one of a node of the acoustic wave, an antinode of the acoustic wave and the wall surface portion, e.g. to determine attachment of particles to a wall.

Thus, using the method effects of such forces may be studied.

In view of the foregoing, herewith a system is provided. The system comprises a sample holder comprising a holding space for holding a sample comprising one or more particles in a fluid medium, wherein the sample holder comprises a wall providing a wall surface portion in the holding space; a signal generator for providing a driving signal, having a signal frequency, a signal amplitude and a signal power, to the sample holder generating a standing longitudinal acoustic wave in the sample holder comprising at least one of a node and an antinode in the holding space, preferably for applying an acoustic force to the particle, the acoustic wave in particular being an ultrasound wave; a controller module for controlling the signal generator and one or more devices for detection and/or measuring data indicative of at least part of the driving signal and/or of the acoustic wave and/or of the sample holder and/or of the sample. The sample holder, signal generator and controller module are operably connectable or connected to generate the acoustic wave in the sample holder.

The system further comprises a computer readable storage medium having computer readable program code embodied therewith, and a processor, preferably a microprocessor, coupled to the computer readable storage medium, wherein responsive to executing the computer readable program code, the processor is configured to perform executable operations comprising:
determining on the basis of the data an acoustic resonance frequency characteristic of the sample holder for the driving signal;
determining a variation in the resonance frequency characteristic of the sample holder;
providing on the basis of the variation a signal indicative of a difference in position of one or more of the particles with respect to at least one of a node of the acoustic wave, an antinode of the acoustic wave and the wall surface portion, in particular at least one of contact and adhesion of one or more of the particles to the wall surface portion.

Such system or at least some embodiment thereof is capable of performing the method disclosed herein, providing at least some of the associated features and benefits.

The sample holder may be included in an electric circuit. The system may then be configured for providing at least part of the driving signal as an electrical signal, preferably an oscillating signal, and for determining at least one property of the driving signal and/or of the sample holder selected from a group consisting of a voltage drop, an impedance, an admittance, a susceptance, a conductance and a signal phase shift.

In such system, the processor may be configured to perform executable operations comprising: determining a value of the selected property at a plurality of signal frequencies in a signal frequency range; and determining on the basis of at least the selected property the resonance frequency and/or a quality factor for the sample holder with respect to the resonance frequency.

Such system facilitates determining the resonance frequency and/or the quality factor, e.g. facilitating including the determination in an automated detection method and/or in a test- and measurement protocol. Also or alternatively, such system facilitates incorporation of several detection devices into the system, e.g. microscopes, since electrical circuitry may provide little or no interference with other detection systems.

In at least one embodiment, the wall surface portion is provided with a functionalized wall surface layer and/or the one or more particles comprise a cellular body.

The system may comprise a sample holder assembly comprising a plurality of the sample holders as described herein and/or a sample holder comprising a plurality of the holding spaces as described herein each comprising one or more of the wall surface portions described herein.

The system may be configured for determining a respective acoustic resonance frequency characteristic of each of the plurality of the sample holders and/or for each of the plurality of the holding spaces; determining a variation in the respective resonance frequencies characteristic of each of the plural sample holders and/or for each of the plural holding spaces; and providing a signal indicative of a difference in position of one or more of the particles with respect to at least one of a node of the respective acoustic wave, an antinode of the respective acoustic wave and respective the wall surface portion, in particular at least one of contact and adhesion of one or more of the particles to the respective wall surface portion.

Thus, a batch of samples and/or of sample holders and/or of holding spaces may be studied.

Further, a computer program or suite of computer programs is provided comprising at least one software code portion or a computer program product storing at least one software code portion, the software code portion, when run on a computer system, being configured for executing the method steps according any embodiment discussed herein.

### BRIEF DESCRIPTION OF THE DRAWINGS

The above-described aspects will hereafter be more explained with further details and benefits with reference to the drawings showing a number of embodiments by way of example.

FIG. 1 shows a schematic drawing of an embodiment of a system in accordance with the present concepts;

FIG. 2 shows a cross section of a sample holder;

FIG. 2A shows a detail of the sample holder of FIG. 2 indicated with “IIA”;

FIG. 3 shows a simplified schematic circuit of an electrical setup of an embodiment of a manipulation system in accordance with the present concepts;

FIG. 3A shows a complex phase diagram of the system of FIG. 3;

FIG. 4 shows simulated data of, from top to bottom admittance, conductance and susceptance versus frequency, and a resonance peak having a Full Width at Half Maximum value γ;

FIGS. 5-6 show exemplary measurements of the data indicated in FIG. 4;

FIG. 7 shows exemplary measurements of conductance of a sample holder versus signal frequency for various temperatures;

FIG. 8 shows exemplary measurements of a temperature dependency of the resonance frequency f₀ for a sample holder as a function of temperature at two different days a week apart;

FIG. 9 shows a time dependency of the resonance frequency f₀ of a sample holder for several times of application of an acoustic wave;

FIGS. 10A-10C are microscope images of silica microspheres as sample particles after several acoustic force runs;

FIG. 11 shows the behaviour of the resonance frequency f₀ with respect to signal power for subsequent force runs;

FIGS. 12A-12C and FIGS. 13A-13C schematically show a sample holder with different numbers of sample particles;

FIGS. 14A-15B show similar experiment results to FIGS. 10A-11, using biological cells instead of silica microspheres;

FIG. 16 shows an alternative embodiment, comprising a gyroscope provided with a sample holder;

FIGS. 17A and 17B show yet another embodiment, comprising plural holding spacers in a wafer.

### DETAILED DESCRIPTION OF EMBODIMENTS

It is noted that the drawings are schematic, not necessarily to scale and that details that are not required for understanding the present invention may have been omitted. The terms “upward”, “downward”, “below”, “above”, and the like relate to the embodiments as oriented in the drawings, unless otherwise specified. Further, elements that are at least substantially identical or that perform an at least substantially identical function are denoted by the same numeral, where helpful individualized with alphabetic suffixes.

Further, unless otherwise specified, terms like “detachable” and “removably connected” are intended to mean that respective parts may be disconnected essentially without damage or destruction of either part, e.g. excluding structures in which the parts are integral (e.g. welded or moulded as one piece), but including structures in which parts are attached by or as mated connectors, fasteners, releasable self-fastening features, etc. The verb “to facilitate” is intended to mean “to make easier and/or less complicated”, rather than “to enable”.

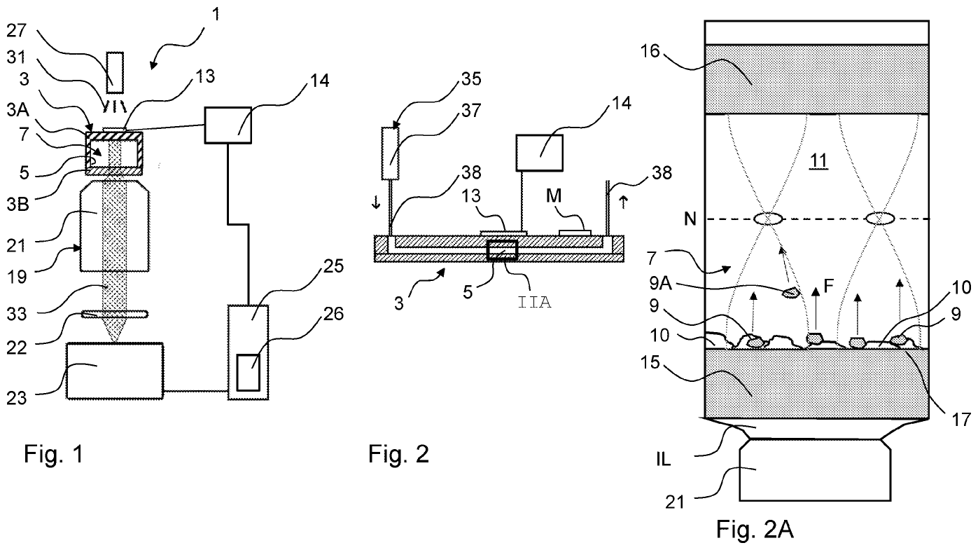

FIG. 1 is a schematic drawing of an embodiment of a system 1 in accordance with the present concepts, FIG. 2 shows a cross section of a sample holder for use in the system 1 and FIG. 2A is a detail of the sample holder of FIG. 2 as indicated with “HA”.

The system 1 comprises a sample holder 3 comprising a holding space 5 for holding a sample 7 comprising one or more biological cellular bodies 9 in a fluid medium 11 as exemplary particles of interest. It is noted that also, or alternatively, other types of particles like microspheres could be used. The fluid preferably is a liquid or a gel. The system 1 further comprises an acoustic wave generator 13, e.g. a piezo element, connected with the sample holder 3 to generate an acoustic wave in the holding space 5, possibly exerting a force on the sample 7 and cellular bodies 9 in the sample 7. The acoustic wave generator 13 is connected with an optional controller module 14 and power supply, here being integrated.

The sample holder 3 comprises a wall 15 providing the holding space 5 with wall surface portion 17 to be contacted, in use, by part of the sample 7. The wall surface portion may be functionalized. A further wall, e.g. opposite wall 16, provides another wall surface portion 17 to be contacted, in use, by part of the sample 7. One or both wall surface portions may be provided with a functionalized wall surface portion.

The shown manipulation system 1 optionally comprises a microscope 19 with an objective 21 and a camera 23 connected with a computer 25 comprising a controller and a memory 26. The computer 25 may also be programmed for tracking one or more of the cellular bodies based on signals from the camera 23 and/or for performing microscopy calculations and/or for performing analysis associated with (superresolution) microscopy and/or video tracking, which may be sub-pixel video tracking. The computer or another controller (not shown) may be connected with other parts of the system 1 (not shown) for controlling at least part of the microscope 19 and/or another detector (not shown). In particular, the computer 25 may be connected with one or more other parts of the system such as the acoustic wave generator 13, the power supply thereof, the controller 14 thereof (both as shown in FIG. 1), the light source, a temperature control, sample fluid flow control, etc. (none shown).

The system further optionally comprises a light source 27. The light source 27 may illuminate the sample 7 using any suitable optics (not shown) to provide a desired illumination intensity and intensity pattern, e.g. plane wave illumination, Köhler illumination, etc., known per se. Here, in the system light 31 emitted from the light source 27 is directed through the acoustic wave generator 13 to (the sample 7 in) the sample holder 3 and sample light 33 from the sample 7 is transmitted through the objective 21 and through an optional further lens 22 and/or further optics (not shown) to the camera 23. The objective 21 and/or lens 22 and the camera 23 may be integrated; the objective and/or lens may comprise plural lens elements. In an embodiment, two or more optical detection tools, e.g. with different magnifications and/or components related to spectral and/or polarization properties, may be used simultaneously for detection of sample light 33, e.g. using a filter and/or a beam splitter,

In another embodiment, not shown but discussed in detail in WO 2014/200341, the system comprises a partially reflective reflector and light emitted from the light source is directed via the reflector through the objective and through the sample, and light from the sample is reflected back into the objective, passing through the partially reflective reflector and directed into a camera via optional intervening optics. Further embodiments may be apparent to the reader.

The sample light 33 may comprise light 31 affected by the sample (e.g. scattered and/or absorbed) and/or light emitted by one or more portions of the sample 7 itself e.g. by chromophores and/or fluorophores attached to the cellular bodies 9.

Some optical elements in the system 1 may be at least one of partly reflective, dichroic (having a wavelength specific reflectivity, e.g. having a high reflectivity for one wavelength and high transmissivity for another wavelength), polarization selective and otherwise suitable for the shown setup. Further optical elements e.g. lenses, prisms, polarizers, diaphragms, reflectors etc. may be provided, e.g. to configure the system 1 for specific types of imaging and/or microscopy.

The sample holder 3 may be formed by a single piece of material with a channel inside, e.g. glass, injection molded polymer, etc. (not shown) or by fixing different layers of suitable materials together more or less permanently, e.g. by welding, glass bond, gluing, taping, clamping, etc., such that a holding space 5 is formed in which the fluid sample 7 is contained, at least during the duration of an experiment. As shown in FIGS. 1 and 2, the sample holder 3 may comprise a part 3A that has a recess being, at least locally, U-shaped in cross section and a cover part 3B to cover and dose (the recess in) the U-shaped part providing an enclosed holding space 5 in cross section. A monolithic sample holder, at least at the location of the acoustic wave generator 13, may be preferred over an assembled sample holder for one or more of improving acoustic coupling, reducing losses, improving signal to noise and preventing local variations.

As shown in FIG. 2, the sample holder 3 is connected to an optional fluid flow system 35 for introducing fluid into the holding space 5 of the sample holder 3 and/or removing fluid from the holding space 5, e.g. for flowing fluid through the holding space (see arrows in FIG. 2). The fluid flow system 35 may comprise a manipulation and/or control system, possibly associated with the controller 14 and/or the computer 25. The fluid flow system 35 may comprise one or more of reservoirs 37, pumps, valves, and conduits 38 for introducing and/or removing one or more fluids, sequentially and/or simultaneously. The sample holder 3 and the fluid flow system 35 may comprise connectors, which may be arranged on any suitable location on the sample holder 3, for coupling/decoupling without damaging at least one of the parts 3, 35, and preferable for repeated coupling/decoupling such that one or both parts 3, 35 may be reusable thereafter. Further, an optional machine-readable mark M or other identifier is attached to the sample holder 3, possibly comprising a memory.

FIG. 2A is a schematic of a number of cellular bodies 9 in the sample holder 3 of FIG. 2. Part of the wall 15 of the sample holder 3 is optionally provided with a functionalized wall portion 17, e.g. an area of the wall being covered with biological cells 10 of a different type than the cellular bodies 9 to which the cellular bodies of interest 9 may adhere. Also shown is part of the microscope lens 21 and an optional immersion fluid layer IL for improving image quality.

On providing a periodic driving signal to the acoustic wave generator 13 a longitudinal acoustic wave is generated in the sample holder 3 directed into the holding space 5. Such compression wave exerts a force on sample particles, e.g. the cellular bodies 9, having a different compressibility and/or density than the surrounding sample fluid 11. In particular, the frequency of the acoustic wave is adjusted so that a standing acoustic wave is generated in the sample holder 3. The signal may be selected, as indicated, such that an antinode of the standing wave is generated at or close to the wall surface portion 17 and a node N of the standing wave away from the wall surface portion 17, generating a local maximum force F on the bodies 9 at or near the surface 17 towards the node N which may be sufficient to displace one or more of the cellular bodies towards the node N (see 9A). Thus, application of the signal may serve to probe adhesion of the bodies 9 to the surface and/or any functionalized layer on the surface portion 17 in dependence of the acoustic force.

In an example an optimal force generation for particular studies may be achieved by selecting acoustic cavity parameters and the frequency/wavelength of the acoustic wave in order to create a maximum pressure gradient at the functionalized wall surface, e.g. by ensuring that the distance from the wall surface to the acoustic node is approximately ¼ wavelength.

As an alternative, the frequency of the acoustic wave could be adjusted so that a node is formed close to the wall surface and an antinode away from the surface portion to provide a local maximum force F on the bodies 9 towards the surface portion (not shown), rather than away from it.

Note further that dependent on the relation between the acoustic wavelength and the size of the sample holder, plural nodal planes N may be formed in the holding space.

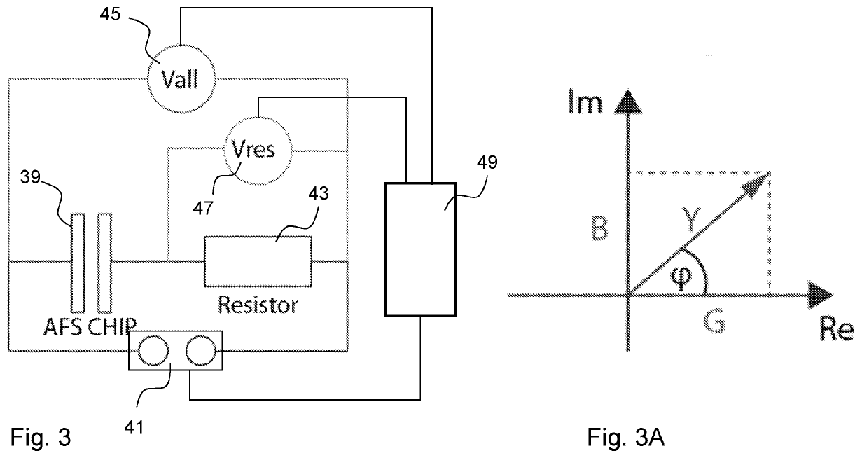

FIG. 3 shows a simplified schematic circuit of an electrical setup of the system 1. Here, the sample holder 3 and acoustic wave generator 13 are considered together as a functional unit “AFS Chip” indicated at 39, having an impedance Z. A power supply 41 is provided for generating the periodic driving signal, having a signal frequency, a signal amplitude and a signal power. The power supply 41 is connected to the chip 39 in series with a reference resistor 43. Voltage measurement devices 45, 47 for measuring Vₐₗₗ and Vᵣₑₛ are provided as indicated. The power supply 41 and voltage measurement devices 45, 47 are connected to a controller 49, which may comprise an analog-to-digital converter (ADC) and/or which may be connected with, or be part of, the controller 14 and/or the computer 25 shown in FIGS. 1 and 2. V_{chip}, the voltage across the acoustic wave generator, can then also be calculated as V_{chip}=Vₐₗₗ−Vᵣₑₛ.

When providing an oscillating driving voltage Vᵢₙ by the power supply 41 to the chip 39, a phase difference φ between Vₐₗₗ and Vᵣₑₛ will occur, which may be measurable. The following values may be determined (see also the complex phase diagram in FIG. 3A):

Impedance: |Z|=(V_{chip})Rᵣₑₛ/Vᵣₑₛ

Admittance: |Y|=1/|Z|

wherein

Complex admittance: Y=|Y|exp(−jφ)=G+jB

Susceptance: B=|Y|sin(−φ)

Conductance: G=|Y|cos(φ)

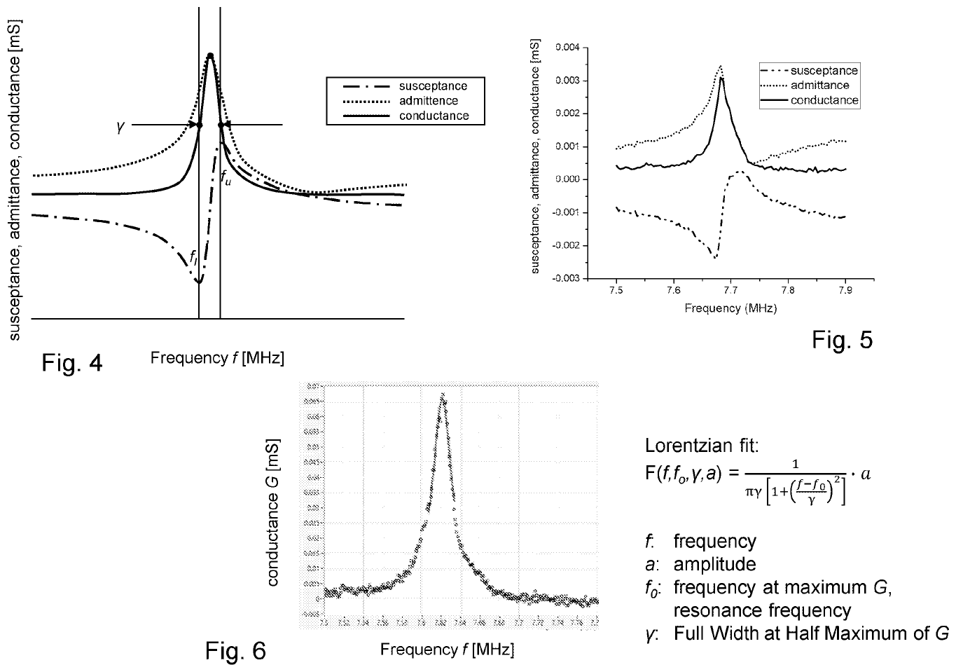

A frequency sweep, e.g. with a width of 0.5 MHz or some other suitable width, may be performed around a calculated resonance frequency. A resonance frequency f₀ is identified at the frequency f where the conductance G is at a maximum, impedance |Z| is at a minimum, admittance |Y| is 0. Such case is indicated in FIG. 4 showing from top to bottom admittance, conductance and susceptance versus frequency; the resonance peak has Full Width at Half Maximum value FWHM. FIGS. 5 and 6 show measured data of an exemplary sample holder. The resonance frequency and/or FWHM-value of the resonance peak may be determined from determining extreme values fₗ, fᵤ of the susceptance B and/or from fitting the resonance peak w suitable function, e.g. a Lorentzian fit as shown in FIG. 6 and the corresponding fit-equation adjacent the graph.

The quality factor Q of the chip resonance peak is determined as:

Quality factor: Q=f₀/(fₗ−fᵤ)=f₀/FWHM=f₀/γ

Thus, by measuring a suitable combination of properties of the sample holder and/or the system such as voltage, resistance, phase difference, impedance, admittance, conductance and/or susceptance for various frequencies and determining values thereof, the resonance frequency and/or the quality factor of the sample holder may be determined; note that presence of a sample in the sample holder and any properties of the sample may affect the resonance frequency and quality factor but these will likewise be determined.

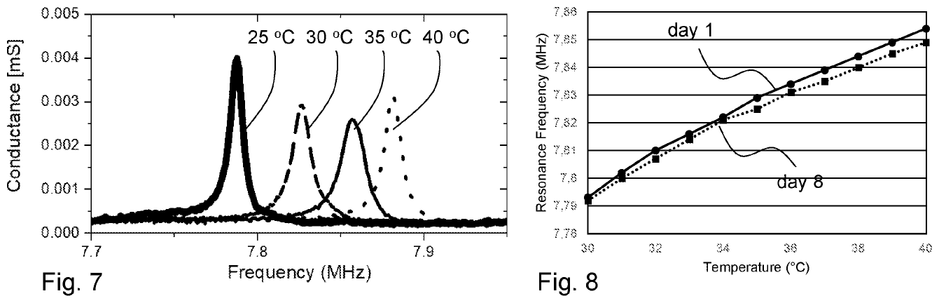

FIG. 7 shows that for one studied chip the position and shape of the conductance peak, meaning the resonance frequency f₀ and the system behavior at a frequency at or near the resonance frequency f₀, is strongly dependent on temperature: f₀=f₀(T). Temperature changes may e.g. be due to one or more of environment, system, temperature control and force generation. This effect may have to be taken into account when using a resonance frequency characteristic for accurate determination of a difference in position of one or more of the particles with respect to at least one of a node of the acoustic wave, an antinode of the acoustic wave and the wall surface portion, see below. The momentary resonance frequency may be determined by frequency modulating the driving signal about the (expected) resonance frequency and detecting the conductance as a function of the modulated driving signal frequency.

FIG. 8 shows a temperature dependency of the resonance frequency f₀ for one studied chip as a function of temperature at two different days a week apart. Clearly, the overall temperature dependency of the resonance frequency f₀(T) is the same, but the values differ. Several causes could attribute to such effects including aging of the sample and/or sample holder. Associated therewith, the quality factor may depend on temperature and/or time.

Thus, it may be beneficial to determine a reference value and/or reference behavior for a particular sample holder to be used for a sample. The reference may comprise determining a baseline curve under controlled conditions, e.g. performing one or more measurements with a test sample comprising a fluid sample medium but without particles in the sample. Any embodiment of the method may then comprise comparing one or more determined values and/or behaviors for the sample of interest (including one or more particles) with a suitable value from the reference. This may improve accuracy and/or reliability of the method.

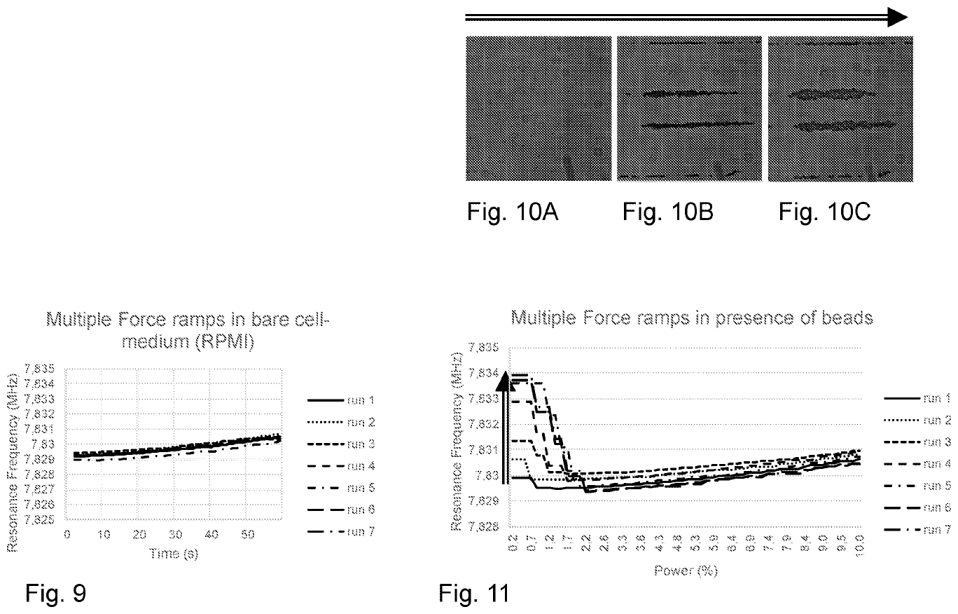

FIGS. 9-11 show experimental results of method steps of an embodiment using a sample holder comprising holding space with a glass wall without surface layer. The sample holder was provided with a piezoelectric signal generator for generating an acoustic wave in the sample holder and the holding space. The sample holder was provided with a sample comprising a cell culture medium (Roswell Park Memorial Institute (RPMI) 1640 Medium) as fluid sample medium. The sample temperature was around 37 degrees Celsius.

A resonance frequency f₀ of the sample holder comprising the sample was determined and a driving signal was provided seven (7) times to the sample holder for a period of time during which the signal power was ramped up linearly from 0.2% to 10% of the maximum rated power for the chip. During each such “force ramp”, or: “run”, one or more resonance frequency characteristics were determined and variations in each characteristic were determined.

E.g., FIG. 9 shows a dependency of the resonance frequency f₀ of the sample holder comprising the sample as a function of time of application of the driving signal (and thus of the acoustic wave) to the sample holder for 7 such of applications of the driving signal (i.e. seven equal force ramps). The resonance frequency behaviors of each time of application of the driving signal are clearly substantially the same, within some measurement error and/or a small offset between the curves possibly caused by changes in environmental temperature. The variations of the resonance frequency with application time within each run are thought to result from heating of the sample holder and sample due to energy deposition into the sample and sample holder by the acoustic wave.

FIGS. 10A-10C are microscope images of another method step using the sample holder. Different from the previous discussion associated with FIG. 9, the sample comprised silica beads with an average diameter of 16.1 micrometer (standard deviation 0.1 micrometer) as particles in the fluid sample medium. The compressibility and density of the beads differs from that of the sample fluid RPMI. Hence, an acoustic wave in the sample may exert an acoustic force on the beads.

Prior to measurement, the sample comprising the beads was flushed into the holding space. After flushing in, the beads were allowed to sink to the surface providing a random distribution, see FIG. 10A. Next, the driving signal was provided to the sample holder seven times in force ramps like those of FIG. 9, although more or less times could have been used. Different from FIG. 9, also, each force ramp was performed over 6 seconds.

During each force ramp the beads were pushed up from the glass wall surface portion towards an axial acoustic node, in a nodal plane approximately half way between the glass wall surface 15 and opposite wall 16, as indicated by N in FIG. 2A. Between force ramps the driving signal was turned off and the beads were allowed to sink back to the surface of the wall. However, even though FIG. 2A only illustrates the presence of an axial nodal plane, substantial lateral variations in the force distribution on the wall surface are present in the actual chip. As a result, every time the beads were pushed towards the nodal plane N they had the tendency to also aggregate in lateral acoustic nodes.

FIG. 10B shows the positions of the particles after three force ramps and FIG. 10C shows the positions of the particles after all seven force ramps. From these figures, it will be evident that after three runs (FIG. 10B) many beads have aggregated into four lines in the field of view with most beads concentrated in the center two lines to which the force appeared to be the highest. After all seven runs (FIG. 10C) even more beads have aggregated into the central lateral nodes. It appears that a number of beads have been drawn into these nodes also from regions outside of the field-of-view.

During each of the force ramps, the resonance frequency of the sample holder comprising the sample was measured, and (momentary) variations in the resonance frequency f₀ were determined by frequency locking.

FIG. 11 shows the behavior of the resonance frequency f₀ with respect to the signal power for each of the seven force ramps (“run 1” . . . “run 7”), determined in addition to determination of the positions of the particles using the microscope (FIGS. 10A-10C; compare the wide arrows). Clearly, the resonance frequency f₀ of the sample holder comprising the sample varies with the driving signal power and with the number of times of providing the driving signal. From FIG. 11, it is clear that during the first part of each force ramp the resonance frequency sharply drops, as the beads are forced from the wall surface portion and towards the axial node (compare FIGS. 10A-10C).

In particular, starting from a random distribution of beads before application of a force ramp (FIG. 10A) the beads are not only lifted from the wall surface but also aggregate to specific regions of the chip as a result of lateral components in the acoustic force field (cf. FIGS. 10B, 10C), possibly associated with lateral acoustic standing wave nodes. After each force ramp the beads fall back towards the wall surface portion as a new starting position. Thus, after several force ramps, the aggregation becomes more pronounced and beads may accumulate in the field of view from locations outside of the field of view (compare FIGS. 10B and 10C).

The concentration of particles in regions of local extreme acoustic forces or force gradients may cause concentration of particles in regions of maximum acoustic coupling from the acoustic wave generator into the sample holder, and thus maximum coupling between the particles and the acoustic wave.

With respect to the variation between the successive force ramps 1-7 it may be seen in FIG. 11 that for the first run the drop in resonance frequency is only around 385 Hz (49 ppm) while for subsequent runs (as more beads aggregate to the high force regions, or high force gradient regions, of the chip) the drop in frequency gets progressively more pronounced until it is around 4500 Hz (575 ppm) for run 7.

These results indicate that not only the presence of beads on the wall surface, and the distance of the beads from the wall surface influence the resonance frequency, but also that the position of the beads with respect to the lateral force field variations in the chip influences the resonance frequency.

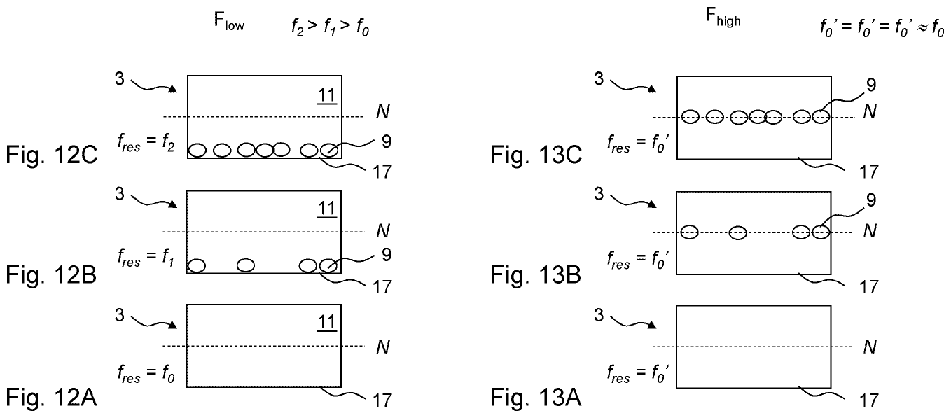

Note that, once all beads have been lifted from the wall surface portion (at a driving signal power of about 2.5% or higher of the rated power), the resonance frequency behavior with respect to the driving signal power is the same or at least substantially the same as for the reference measurements (FIG. 9). Thus, the density of beads in the acoustic node was found not to influence the resonance frequency significantly. It is considered that this may be associated with the relatively low acoustic intensity at or near an acoustic node. Conversely, as also indicated above with respect to FIGS. 10A-11, concentrating the beads in regions of comparably high forces and/or high force gradients (as a result of multiple force ramps) tends to increase the difference between the resonance frequency at low powers, associated with the beads being in contact with the wall surface portion, and the resonance frequency at comparably high powers, associated with all beads being separated from the wall surface portion. This latter effect is indicated in FIGS. 12A-12C and FIGS. 13A-13C: each of the figures schematically shows a sample holder 3 in different situations. FIGS. 12A-12C show three different situations at low acoustic force F_{low}, from bottom to top: FIG. 12A: no particles on the wall surface 17, resonance frequency fᵣₑₛ=f₀. FIG. 12B: low density of particles 9 on the wall surface 17, resonance frequency fᵣₑₛ=f₁>f₀. FIG. 12C: high density of particles 9 on the wall surface 17, resonance frequency fᵣₑₛ=f₂>f₁. FIGS. 13A-13C show likewise that after application of sufficient acoustic force F_{high}, when all the particles are in the acoustic node, the resonance frequency fᵣₑₛ is the same f₀′ no matter the density of the particles in the node. Please note that the resonance frequency f₀′ may differ from the resonance frequency at no or low acoustic power due to temperature changes and/or other experimental conditions.

Thus, differences in position of the particles may be determined on the basis of the variation relation between the resonance frequency and the driving signal power as well as between the resonance frequency and the number of times of providing the driving signal (i.e. providing an acoustic force).

It is noted that, not shown, not only the resonance frequency varies with a change in position of the particles relative to at least one of a node of the acoustic wave, an antinode of the acoustic wave and the wall surface portion, but that also or alternatively the FWHM-value of the resonance peak and/or the resonance quality factor tend to differ. Such differences may be detectable and used for determination of (behavior of) variations in position and/or attachment, etc., of one or more particles to the wall surface portion.

Measurements may further be performed as a function of one or more sample preparation steps, incubation steps, reaction mechanisms and the like, which may e.g. comprise temperature variations.

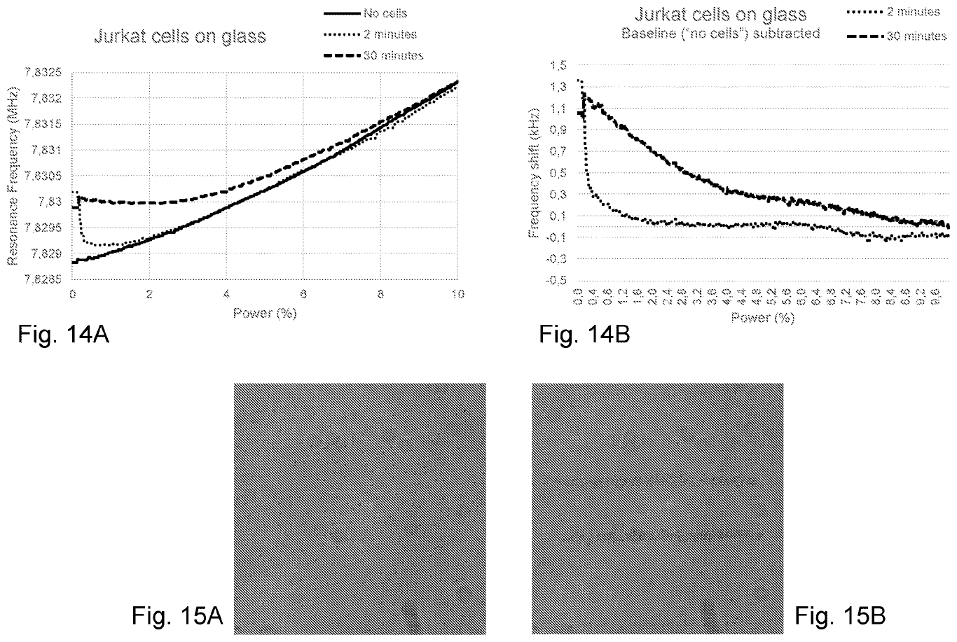

FIGS. 14A-15B shows a similar experiment as discussed above with respect to FIGS. 9-13C. Here, however, instead of silica beads, Jurkat cells were used as particles in the sample further comprising PRMI as a sample fluid. Jurkat cells are cells of a standard immortalized T-cell line which have a known tendency to stick to glass if they are allowed to incubate for sufficient time.

For the experiments, a sample holder comprising a holding space and having a wall providing a wall surface portion in the holding space was provided, further comprising a signal generator for providing a driving signal to the sample holder generating an acoustic wave in the sample holder for applying an acoustic force to the cells.

Referring to FIG. 14A, first, as a reference, a sample of RPMI as a fluid medium was provided in the holding space, without cells. Then, using the signal generator a driving signal was applied to the sample holder in a force ramp as explained above, while determining an acoustic resonance frequency characteristic of the sample holder to provide a reference frequency response; see FIG. 14A (drawn line). The resonance frequency was found to rise substantially monotonically attributed to heating up of the sample holder and sample due to increased energy dissipation at higher signal powers.

Next, Jurkat cells (Clone E6-1 (ATCC® TIB-152™) at a concentration of about 1 million cells/ml where flushed in into the holding space and allowed to incubate on the glass wall surface for two minutes. Incubation time was counted from the moment of stopping the flow. FIG. 15A is a microscope image of the cells after flushing in. The cells are randomly distributed over the field of view, visible as dark spots in contact with the glass.

After incubation, the driving signal was applied again to provide an acoustic standing wave force to the cells to urge them towards the node in the holding space. As before, the driving signal power and thus the acoustic force were ramped up. During application of the signal the acoustic resonance frequency and any variation therein were determined as a function of time and signal power.

With the Jurkat cells in contact with the glass at a density of approximately 650 cells in the field of view of the microscope (here being ca 1.7 mm×1.7 mm), the frequency of the acoustic resonance was found to shift from about 7.8288 to 7.8302 MHz, a shift of 1.4 kHz, at lowest driving signal powers; see FIG. 14A, dotted line. Upon increasing the acoustic force, cells were lifted off from the glass wall surface portion and the resonance frequency characteristic of the sample holder returns to the reference signal at a power of about 2% rated power of the chip.

Because the cells have been allowed to incubate for only 2 minutes between flushing in and application of the acoustic force, they are only moderately bound to the glass wall surface portion in the holding space and most cells come off from the surface at only very low forces. The acoustic resonance frequency drops back to the baseline RPMI curve within the first 200 pN of reference force; the reference force for the chip is, as an option, defined as the expected force at a given power level for the chip concerned, according to a calibration on silica beads with known acoustic properties. Also or alternatively the true force on the cells can be determined and/or estimated.

When all cells have lifted off the wall and reached the acoustic node, interaction between the acoustic field and cells is minimal. Therefore the resonance frequency is not, or hardly, affected compared to the reference experiment (see also the discussion above).

FIG. 15B shows that the cells have indeed come loose from the wall surface portion and moreover that they have become spatially redistributed in relation with the acoustic force distribution in the holding space, just like the beads discussed above. Such optical measurement may be used for various other forms of detection and/or study.

Thereafter, the previous experiment was repeated using a longer incubation time of about 30 minutes. The measurement results are also indicated in FIG. 14A (dashed line).

Again, the initial resonance frequency shift with respect to the baseline is similar as for the short incubation time. Note that at a signal power below 0.3% the signal-to-noise ratio of the resonance frequency measurement was found to be too low to estimate the resonance frequency reliably with the used measurement setup. From a comparison of the resonance frequency behavior of the two experiments using cells, it will be evident that after longer incubation time, the cells stick much more strongly to the glass wall surface than if allowed only a short incubation time and the cells come off the wall surface portion only at much higher signal powers/acoustic forces.

Thus, it is show that an adhesion strength of the cells to the wall surface can be sensed and may be determined reliably, wherein microscopic imaging and/or video analysis may not be needed.

For comparison and possible increased sensitivity, FIG. 14B shows the same data of the measurement results of the experiments comprising cells in the sample as in FIG. 14A but here the resonance frequency baseline obtained without cells is subtracted from the data obtained with the cells at 2 and 30 minutes incubation time respectively. As a result. FIG. 14B now directly shows a resonance frequency shift as a function of the applied signal power (associated with the acoustic force in the sample holder) which correlates with the detachment of the cells from the wall surface portion. Such curve can therefore be interpreted directly as an avidity curve.

It is noted that the results determined with the above-described experiments may obviate video-based detection methods which may be demanding in terms of required resources (camera, lenses, alignment and/or focusing, computational power etc.). However, both optical methods and the presently provided method may be used in combination for improved sensitivity and/or performing different determinations in parallel.

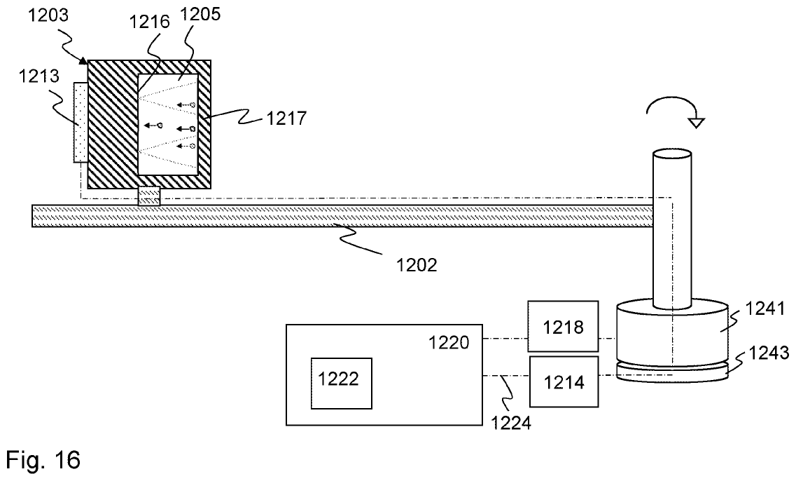

FIG. 16 shows an alternative embodiment of the present concepts. Here, part of the sample holder is rotated and a centrifugal force is used for urging cellular bodies away from, or towards, a wall surface portion, instead of, or in addition to, an acoustic force. The signal generator and the generated acoustic wave, and associated determinations of resonance frequency characteristics may thus be used for sensing and detection only, or at least predominantly.

In particular, FIG. 16 schematically depicts a rotary arm 1202 that is rotatable mounted onto a rotor system that includes a rotor 1241 and a rotary joint 1243. A sample holder 1203 comprising a holding space 1205 for holding a sample comprising one or more particles in a fluid medium is mounted to the arm 1202. The sample holder 1203 comprises a wall surface portion 1217 in the holding space 1205. The sample holder 1203 further comprises an acoustic wave generator 1213 connected to a signal generator 1214. A rotor controller 1218 may be used to control the rotor system to spin the rotary arm in a circular motion so that a centrifugal force is exerted onto the particles, e.g. biological cells. The system may be connected to a computer 1220 that includes a processing module 1222 that is configured to control the rotor controller and the signal generator 1214. Further, when in use, the computer may receive signals from the signal generator 1213 while a force ramp is exerted onto the particles. Further, the processing module 1222 may be configured to detect and track the resonance frequency of the sample holder in a similar way as described with reference to FIGS. 4.6 above, possibly associated with modulating the driving signal and/or locking techniques.

In the configuration sketched in FIG. 16, an acoustic antinode is located at or near the wall surface 1217 in radial inward direction, i.e. opposite of the sample holder 1203 compared to the acoustic wave generator 1213, e.g. by selection of the acoustic frequency. However, the acoustic wave generator 1213 could be arranged on the radial inside. An acoustic antinode or high pressure gradient is arranged at the (functionalized) wall surface 1217. An acoustic resonance frequency of the sample holder may be determined as set out above. As before, particles bound to the wall surface 1217 of the holding space 1205 cause a detectable shift in the resonance frequency.

Upon rotation of the arm 1202, particles that come off the wall surface 1217 travel to the opposite wall surface 1216 under the centrifugal force. Particles leaving the wall surface portion cause that the resonance frequency of the sample holder 1203 comprising the sample approaches the resonance frequency of the sample holder 1203 per se. Since there is an acoustic node on the radially outward side of the holding space, released cells accumulating at the outward side no longer contribute to the resonance frequency shift, or at least they contribute less than cells still adhering to the wall surface portion 1217. A change in resonance frequency can thus be used to measure binding forces of particles to the wall surface portion 1217.

As may be obvious to the reader the acoustic field could also be reversed (i.e. with an acoustic node at the wall surface and an antinode or high gradient region at the opposite wall). In that case the detachment of particles on the radial inward side and accumulation of particles on the outward side would lead to the opposite shift in resonance frequency by virtue of an increased interaction strength.

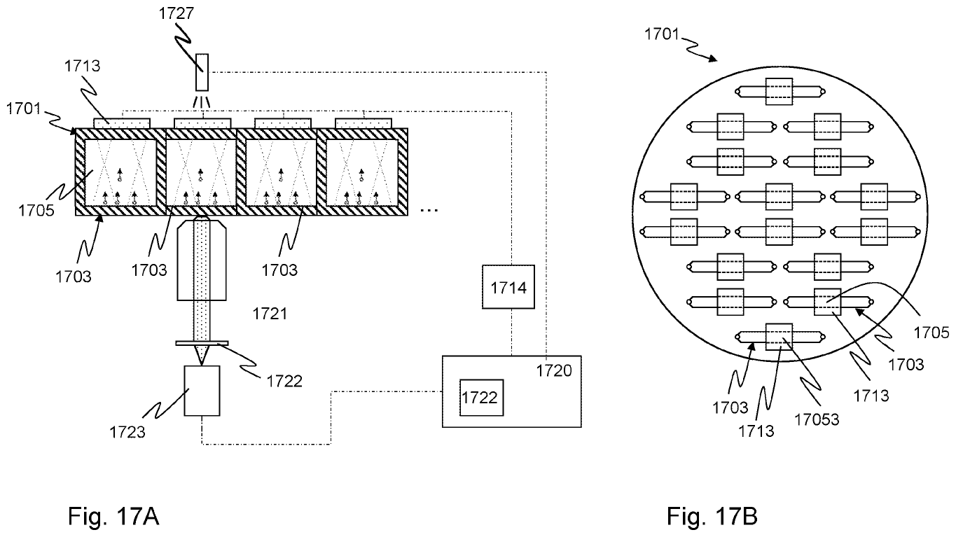

FIGS. 17A and 17B show yet another embodiment: a wafer 1701 is shown implementing 16 individual sample holders 1703, each comprising a holding space 1705 and, as an option, each being provided with its own acoustic wave generator 1713. One or more, preferably all, sample holders 1703 may have channel inlets and outlets from one side and they may be essentially independent chips. One or more of the sample holders 1703 may, also or alternatively, be part of an integrated microfluidics circuit on the wafer 1701. The acoustic wave generators 1713 of at least some of the sample holders 1703 may be connected to individual signal generators and/or to a common signal generator for application of an acoustic wave in the holding space of the respective sample holder 1703 and/or to a computer 1720 that includes a processing module 1722 for detecting and/or processing resonance frequency data.

Such embodiment makes use of the fact that acoustic resonators do not require expensive components and can be easily and cost effectively produced in parallel (e.g. on wafers). This allows scaling up the throughput of a measurement system without adding much cost.

As an option, however, a microscopy system may be used comprising an optional light source 1727, an objective 1721, an optional tube lens 1722 and an imaging sensor 1723 may be used to monitor the particles in one or more of the chips.

The disclosure is not restricted to the above described embodiments which can be varied in a number of ways within the scope of the claims.

For instance, various embodiments may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored.

Further, elements and aspects discussed for or in relation with a particular embodiment may be suitably combined with elements and aspects of other embodiments, unless explicitly stated otherwise.

1. A method comprising:
providing a sample holder comprising a holding spaced for holding a sample comprising one or more particles in a fluid medium, wherein the sample holder comprises a wall providing a wall surface portion in the holding space, and
providing a signal generator for generating an acoustic wave in the sample holder;
providing using the signal generator a driving signal, having a signal frequency (f), a signal amplitude and a signal power (P), to the sample holder generating a standing longitudinal acoustic wave in the sample holder comprising at least one of a node (N) and an antinode in the holding space; and
determining an acoustic resonance frequency characteristic (f₀, Q) of the sample holder for the acoustic wave;
wherein the method further comprises:
providing a sample comprising one or more particles in a fluid medium in the holding space, in particular being in contact with the wall surface portion;
determining a variation in the resonance frequency characteristic (f₀, Q) of the sample holder;
determining on the basis of the variation a difference in position of one or more of the particles with respect to at least one of a node of the acoustic wave, an antinode of the acoustic wave and the wall surface portion, in particular at least one of contact, attachment and adhesion of one or more of the particles to the wall surface portion.

2. The method according to claim 1, comprising providing the driving signal a number (n) of times,
wherein determining a variation in the resonance frequency characteristic (f₀, Q) of the sample holder comprises
determining a variation relation between the resonance frequency characteristic (f₀, Q) of the sample holder and at least one of time, the signal power (P), and the number (n) of times of providing a driving signal; and
wherein the method comprises determining on the basis of the variation relation the difference in position of one or more of the particles.

3. The method according to claim 1, comprising changing at least part of the sample and/or allowing at least part of the sample to change as a function of at least one of time, temperature, illumination, sample composition, and flow of at least part of the sample fluid, thus providing a sample change, and
wherein determining a variation in the resonance frequency characteristic (f₀, Q) of the sample holder comprises
determining a change relation between the resonance frequency characteristic (f₀, Q) of the sample holder and the sample change; and
wherein the method comprises determining on the basis of the change relation the difference in position of one or more of the particles.

4. The method according to claim 1, wherein providing a driving signal comprises including the sample holder in an electric circuit and providing at least part of the driving signal as an electrical signal, and
wherein the method further comprises selecting at least one property of the driving signal and/or the sample holder selected from a group consisting of a voltage drop, an impedance (Z), an admittance (Y), a susceptance (B), a conductance (G) and a signal phase shift (φ);
determining a value of the selected property at a plurality of signal frequencies (f) in a signal frequency range; and
determining on the basis of at least the selected property the resonance frequency (f₀) and/or a quality factor (Q) for the sample holder with respect to the resonance frequency (f₀).

5. The method according to claim 1, comprising superposing a modulation frequency (fₘ) on the signal frequency (f), thus providing a modulated signal frequency,
determining a frequency difference between the resonance frequency (f₀) and at least one of the signal frequency (f) and the modulated signal frequency, and
wherein the method further may comprise:
adjusting the modulation frequency (fₘ) and/or the signal frequency (f) in dependence of the frequency difference.

6. The method according to claim 1, wherein the acoustic wave is a standing wave, and/or
the acoustic wave is oriented perpendicular to the wall surface portion, and/or
the acoustic wave provides a force gradient in a direction away from the wall surface portion into the holding space for urging at least some of the particles towards the wall surface portion and/or for urging at least some of the particles away from the wall surface portion and into the holding space.

7. The method according to claim 1, comprising determining an acoustic force on at least some of the one or more particles at or near the wall surface portion and/or
comprising determining an adhesion strength and/or a detachment force of the one or more particles to the wall surface portion.

8. The method according to claim 1, comprising providing the wall surface portion with a functionalized wall surface layer.

9. The method according to claim 1, wherein at least some of the particles are cellular bodies.

10. The method according to claim 8, wherein one of the cellular bodies and functionalized wall surface layer comprises effector cells and the other one of the cellular bodies and functionalized wall surface layer comprises target cells, in particular one of the cellular bodies and functionalized wall surface layer comprising immune cells such as T-cells and the other one of the cellular bodies and functionalized wall surface layer comprises tumor cells;
wherein the method may further comprise determining a binding characteristic of the effector cells to the target cells.

11. The method according to claim 1, comprising applying a non-acoustic force to the one or more particles, in particular in a direction away from or towards the wall surface portion,
the method in particular comprising rotating at least part of the sample holder and applying a centrifugal force to the one or more particles.

12. A system comprising a sample holder comprising a holding space for holding a sample comprising one or more particles in a fluid medium, wherein the sample holder comprises a wall providing a wall surface portion in the holding space;
a signal generator for providing a driving signal, having a signal frequency (f), a signal amplitude and a signal power (P), to the sample holder generating a standing longitudinal acoustic wave in the sample holder comprising at least one of a node (N) and an antinode in the holding space;
a controller module for controlling the signal generator;
one or more devices for detection and/or measuring data indicative of at least part of the driving signal and/or of the acoustic wave and/or of the sample holder and/or of the sample;
wherein the sample holder, signal generator and controller module are operably connectable or connected to generate the acoustic wave in the sample holder; and
wherein the system further comprises a computer readable storage medium having computer readable program code embodied therewith, and a processor coupled to the computer readable storage medium, wherein responsive to executing the computer readable program code, the processor is configured to perform executable operations comprising:
determining on the basis of the data an acoustic resonance frequency characteristic (f₀, Q) of the sample holder for the driving signal;
determining a variation in the resonance frequency characteristic (f₀, Q) of the sample holder;
providing on the basis of the variation a signal indicative of a difference in position of one or more of the particles with respect to at least one of a node of the acoustic wave, an antinode of the acoustic wave and the wall surface portion, in particular at least one of contact and adhesion of one or more of the particles to the wall surface portion.

13. The system according to claim 12, wherein the sample holder is included in an electric circuit and
wherein the system is configured for providing at least part of the driving signal as an electrical signal,
and determining at least one property of the driving signal and/or of the sample holder selected from a group consisting of a voltage drop, an impedance (Z), an admittance (Y), a susceptance (B), a conductance (G) and a signal phase shift (φ);
wherein the processor is configured to perform executable operations comprising:
determining a value of the selected property at a plurality of signal frequencies (f) in a signal frequency range; and
determining on the basis of at least the selected property the resonance frequency (f₀) and/or a quality factor (Q) for the sample holder with respect to the resonance frequency (f₀).

14. The system according to claim 12, wherein the wall surface portion is provided with a functionalized wall surface layer and/or the one or more particles comprise a cellular body.

15. The system according to claim 12, comprising a sample holder assembly comprising a plurality of the sample holders and/or a sample holder comprising a plurality of holding spaces each comprising a one or more wall surface portions,
the system being configured for
determining a respective acoustic resonance frequency characteristic (f₀, Q) of each of the plurality of the sample holders and/or for each of the plurality of the holding spaces;
determining a variation in the respective resonance frequencies characteristic (f₀, Q) of each of the plural sample holders and/or for each of the plural holding spaces; and
providing a signal indicative of a difference in position of one or more of the particles with respect to at least one of a node of the respective acoustic wave, an antinode of the respective acoustic wave and respective the wall surface portion, in particular at least one of contact and adhesion of one or more of the particles to the respective wall surface portion.

16. A computer program or suite of computer programs comprising at least one software code portion or a computer program product storing at least one software code portion, the software code portion, when run on a computer system, being configured for executing the method steps according to claim 1 in a system according to claim 12.

17. The method according to claim 1, wherein generating the standing longitudinal acoustic wave in the sample holder is for applying an acoustic force to the one or more particles.

18. The method according to claim 17, wherein the acoustic wave is an ultrasound wave.

19. The system according to claim 12, wherein the standing longitudinal acoustic wave in the sample holder is for applying an acoustic force to the one or more particles.

20. The system according to claim 19, wherein the acoustic wave is an ultrasound wave.
